(19) United States
(12) Patent Application Publication
(10) Pub. No.: US 2008/0057112 A1
(43) Pub. Date: Mar. 6, 2008

(54) Oral Products

(75) Inventors: Henry Knoop, Valrico, FL (US); Tara Schaneville, Bonita Springs, FL (US)

Correspondence Address: MCDERMOTT WILL & EMERY LLP, 28 STATE STREET, BOSTON, MA, 02109-1775, US

(73) Assignee: BIOTEC FILMS, LLC, Tampa, FL (US)

(21) Appl. No.: 11/596,630

(22) PCT Filed: May 17, 2005
(86) PCT No.: PCT/US05/17287

§ 371 Date: Oct. 25, 2007

Related U.S. Application Data
(60) Provisional application No. 60/571,949, filed on May 17, 2004.

(51) Int. Cl. A61K 33/00; A61K 9/70; A61P 43/00; B01F 7/00

(52) U.S. Cl. 424/443; 366/279; 424/600

(57) ABSTRACT

An apparatus and method for forming a polymer film and/or oral dosage form having an active, content such as a vitamin, that is a considerable or relatively high proportion of the total dry weight percent without being unpleasant to taste, leaving a bitter after taste, having poor mouth feel and/or being slow to dissolve.

### FIELD OF THE INVENTION

The present invention relates to a method and apparatus for producing oral products containing relatively high levels of actives, and to oral dosage forms and the like.

### BACKGROUND OF THE INVENTION

Oral products such as polymer materials carrying active materials, such as vitamins, are known. However, prior art techniques have produced oral products which are unpleasant to taste, leave a bitter after taste, have poor mouth feel or are slow to dissolve. Particular problems occur when the active content makes up a considerable or high proportion of the total by % wt of the dry composition.

The present invention seeks to provide improved methods and apparatus for producing oral products containing relatively high levels of actives, and to oral dosage forms and the like.

### SUMMARY OF THE INVENTION

According to an aspect of the present invention there is provided an oral dosage form comprising one or more vitamins in an amount greater than 15% by weight of the dry composition. Preferably, the vitamins are present in an amount greater than 20% by weight of the dry composition. Still more preferably, the vitamins are present in an amount greater than 25% by weight of the dry composition. Typically, the vitamins are present in an amount between 25 and 35% by weight of the dry composition. In some embodiments the vitamins exceed 35% by wt.

According to an aspect of the present invention there is provided an oral dosage form comprising one or more vitamins in an amount greater than 30% by weight of the dry composition.

According to an aspect of the present invention there is provided an oral dosage for, and method of preparation thereof, containing at least one vitamin and/or mineral and flavorings sufficient to mask the taste of the vitamin and/or mineral.

According to an aspect of the present invention there is provided an oral dosage form comprising one or more vitamins in an amount greater than 20%, a polymer in amount greater than 25%, by weight of the dry composition, and flavorings sufficient to mask the taste of the vitamins.

According to another aspect of the present invention there is provided an oral dosage form comprising a polymer constituent in an amount between 25 and 35% wt, at least one vitamin and/or mineral in an amount between 25 and 35%, and flavorings in an amount between 7 and 18% wt.

According to an aspect of the present invention there is provided a method and apparatus for preparing any one of the above-mentioned oral products.

According to another aspect of the present invention there is provided a method of forming a polymer film containing vitamins and/or minerals, comprising:

preparing a vitamin emulsion, preparing a polymer base, adding the vitamin emulsion to the polymer base, and casting.

According to another aspect of the present invention there is provided a method of forming a polymer film containing vitamins and/or minerals, comprising: preparing a flavor emulsion, preparing a polymer base, adding the flavor emulsion to the polymer base, and casting.

According to another aspect of the present invention there is provided a method of forming a polymer film containing vitamins and/or minerals, comprising: preparing a vitamin emulsion, preparing a flavor emulsion, preparing a polymer base, adding the vitamin and flavor emulsions to the polymer base, and casting.

According to another aspect of the present invention there is provided a method of forming a polymer film containing at least one vitamin and/or mineral, comprising: preparing a vitamin-flavor emulsion, preparing a polymer base, adding the vitamin-flavor emulsion to the polymer base.

According to another aspect of the present invention there is provided a method of forming a polymer-based film product containing multivitamins and/or minerals, comprising: preparing a vitamin-flavor emulsion, preparing a polymer base, adding the vitamin-flavor emulsion to the polymer base.

According to another aspect of the present invention there is provided a method of forming a polymer-based film product containing multivitamins and/or minerals, comprising: preparing a vitamin-flavor emulsion and preparing a polymer base, adding the vitamin-flavor emulsion to the polymer base, wherein the vitamins are present in an amount between 20 and 40% by wt and one or more flavorings are present in an amount between 5 and 20% by weight of the dry composition. Preferably the polymer constituent is present in an amount between 20 and 40% by weight. Typically, the vitamins amount to about 30% (±5%) by wt, the flavors amount to between 7 and 15% by wt, and the polymer base material amounts to about 30% (±5%) by wt of the dry composition.

Preferably the vitamin(s) are present in an amount greater than 20% wt of the dry composition. More preferably the vitamin(s) are present in an amount greater than 25% wt of the dry composition. Still more preferably the vitamin(s) are present in an amount greater than 30% wt of the dry composition.

Preferably, the film contains flavors in an amount greater than 7% by wt of the dry composition. More preferably the film contains flavors in an amount greater than 10% by wt of the dry composition. Still more preferably the film contains flavors in an amount greater than 13% by wt of the dry composition.

According to another aspect of the present invention there is provided a breath freshening strip.

According to another aspect of the present invention there is provided a method of making a breath freshening strip.

According to another aspect of the present invention there is provided an oral product dissolvable in the mouth or throat or buccal cavity.

According to another aspect of the present invention there is provided an apparatus for forming a polymer film comprising vitamins and/or minerals, the apparatus comprising a first holder and mixer for holding and mixing a polymer base solution; and an emulsifier unit comprising a second holder and mixer for holding and mixing a vitamin-flavor emulsion, and an emulsifier in fluid communication with the second holder to emulsify the emulsion, the first holder in fluid communication with the emulsifying unit to mix together the polymer base solution with the emulsion to form a solution for coating onto a coating surface to form the polymer film. Preferably, the first and second mixers are high shear mixers.

Additional objects, advantages and novel features of the invention will be set forth in part in the description which follows, and in part will become apparent to those skilled in the art upon examination of the following and the accompanying drawings or may be learned by practice of the invention.

### BRIEF DESCRIPTION OF THE DRAWING

For a better understanding of the present invention and as to how the same may be carried into effect reference will now be made, by way of example only, to the accompanying drawing in which:

FIG. 1 is a schematic diagram of apparatus for producing an oral product embodying the present invention.

### DETAILED DESCRIPTION OF THE PREFERRED EMBODIMENTS

The preferred process provides an edible film containing a high concentration of active materials (e.g. vitamins, nutrients, herbals, nutraceuticals) that provides a pleasant taste (with minimal active residual taste), good mouth feel and rapid solubility. This film is created by producing a “vitamin-flavor” emulsion which is then incorporated into a film forming base solution. The vitamin-flavor emulsion and the polymer film base are mixed and conditioned separately in a manner which enables high levels of actives (e.g. vitamins) and high levels of flavors to be introduced to the film base before it is cast and cut to yield individual oral products.

Overview:

In general, the method includes, preparing a vitamin-flavor emulsion, preparing a film base, adding the vitamin-flavor emulsion to the film base, mixing the film base solution and vitamin-flavor emulsion, and casting a film according to predetermined drying parameters.

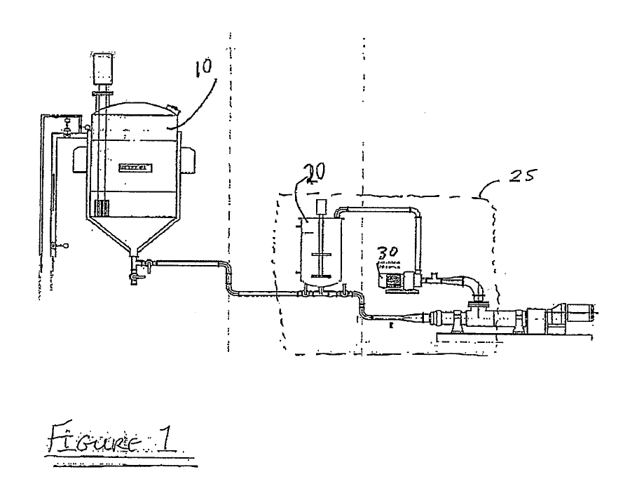

With reference to FIG. 1, suitable apparatus includes a main tank 10, connected to an emulsion tank 20. The emulsion tank 20 is down stream of the main tank and connected in a loop 25 with a commercially available emulsifier 30 (e.g. a Silverson Emulsifier). The apparatus is further connected in the down stream direction to a casting line (not shown), optionally via a filter and/or a casting tank.

The main tank 10 is used mixed to mix the polymer base solution according to procedures and temperatures suitable for type of base film system being used. The main tank 10 is provided with high shear mixing equipment.

The emulsion tank 20 is used to mix the vitamin-flavor emulsion. In general, vitamins are added to water, mixed and emulsified, by being passed through the emulsifier 30 and back into the emulsion tank 10. The emulsion tank 10 is provided with high shear mixing equipment, such as a blade, which imparts high agitation to keep the mix dissolved, namely to keep the droplet size of the emulsified components low.

Exemplary steps in the preferred process are described below.

a. Flavor-Emulsion preparation (in tank 20)
i. Heat water to temperature of about 130 F (120-150 F).
ii. Add vitamins to water under high agitation (includes water soluble and fat soluble vitamins)
iii. Add emulsifiers and stabilizers under high agitation
iv. Add flavor (flavor oil, flavor emulsion, flavor powders) under high agitation
v. Mix and emulsify entire solution for a minimum of 20 minutes
vi. Add vitamin-flavor emulsion to film

b. Mix HPMC base solution (in tank 10)
i. Add water and surfactants to mixing vessel and heat while agitating to 180 F (170-190 F)
ii. Add the HPMC polymer system while agitating at high speed
iii. Mix the said water and polymer system at high speed while cooling the mixture to approx 120 F to form a true solution of all components.
iv. Add the plasticizers and other additives to the mix at less than 120 F.
v. Degas the said mixture and vacuum under low agitation to remove all bubbles;
vi. When the solution get down to 120 F combine the flavor-emulsion mixture from step a.

c. Combine vitamin-emulsion mixture with polymer base

In this case the apparatus facilitates vacuuming the vitamin-emulsion into the polymer base solution.

d. Degassing solution

Degassing occurs while under conditions of vacuum and slow agitation until solution is free of bubbles.

e. Removing said mixture resulting from step (d)

The degassing product is removed from said mixing tank and filtered to remove contaminants and any coarse undissolved ingredients.

f. Casting

The degassed solution mixture is cast on a casting surface;
i. removing said water from said cast, degassed mixture on said casting surface thereby forming said edible film on said casting surface;
ii. wherein said edible film consists essentially of about 4-8% of said water;
   Step (b) above can and be substituted for mixing CMC, Pectin, Alginates, Pullulan, Gum Arabic, Guar gums, Carrageenan and other Starch or polymer systems that are edible and have film forming characteristics.

There follows a number of exemplary and alternative preparations for different film systems.

Solution Preparation—Cellulose Gum (CMC) Base Solutions
1. Heat solution to 140 F.
2. Add CMC under high agitation.
3. Mix thoroughly to confirm complete dissolution and particles are suspended
4. Start slow cooling of solution
5. Slowly add desired ingredients (additives, plasticizers, sweeteners, acids, preservatives, etc.) under high agitation to prevent lumps/gels in solution.
6. If flavor oils are to be added, emulsify flavor oils with emulsifying and/or stabilizing agents to stabilize film. Examples of emulsifiers/stabilizers include Gum Arabic, Xanthan Gum, and Carrageenan. These materials may need to be predispersed/dissolved in water prior to adding to base solution.
7. Add flavor package and continue to mix.
8. Confirm all materials are dispersed/dissolved in solution.
9. De-air solution by vacuuming or let de-air to atmosphere for 12-24 hours.
10. Cool solution to below 100 F prior to casting.

Solution Preparation—Pectin Base Solutions
1. Heat water to 120 F.
2. Add pectin under high agitation, and continue to heat solution to 160 F.
3. Mix thoroughly to confirm complete dissolution and particles are suspended.
4. Start slow cooling of solution to approx 120 F
5. Slowly add desired ingredients (additives, plasticizers, sweeteners, acids, preservatives, etc.) under high agitation to prevent lumps/gels in solution.
6. If flavor oils are to be added, emulsify flavor oils with emulsifying and/or stabilizing agents to stabilize film. Examples of emulsifiers/stabilizers include Gum Arabic, Xanthan Gum, and Carrageenan. These materials may need to be predispersed/dissolved in water prior to adding to base solution.
7. Add flavor package and continue to mix.
8. Confirm all materials are dispersed/dissolved in solution.
9. Degas solution by vacuuming or let degas at atmospheric pressure for 12-24 hours.
10. Cool solution to below 100 F prior to casting.

Solution Preparation—HPMC Base Solutions
1. Heat water to at least 170 F.
2. Add HPMC under high agitation to avoid lumping
3. Mix thoroughly to confirm complete dissolution and particles are suspended
4. Start slow cooling of solution
5. Most materials can be added to the HPMC solution at room temperature. If solution is heated above 130 F, HPMC polymer will come out of solution. The solution needs to be below 130 F for the polymers to remain hydrated and remain in solution.
6. Slowly add desired ingredients (additives, plasticizers, sweeteners, acids, preservatives, etc.) under high agitation to prevent lumps/gels in solution.
7. If flavor oils are to be added, emulsify flavor oils with emulsifying and/or stabilizing agents to stabilize film. Examples of emulsifiers/stabilizers include Gum Arabic, Xanthan Gum, and Carrageenan. These materials may need to be predispersed/dissolved in water prior to adding to base solution.
8. Add flavor package and continue to mix.
9. Confirm all materials are dispersed/dissolved in solution.
10. De-air solution by vacuuming or let de-air to the atmosphere for 12-24 hours.
11. Cool solution to below 100 F prior to casting.

Casting Process

The casting process relates to solution casting, which consists of dissolving one or more synthetic resins in an organic solvent, casting the solution onto a suitable substrate, removing the solvent whereby a film is formed on the carrier, and stripping the film from the carrier. Normally the film is wound into rolls or cut into strips.

In preferred embodiments, the polymer base solution mixed with the vitamin-flavor emulsification is cast and the casting surface might be selected from the group consisting of drums and belts of stainless steel, copper and silicon rubber, and drums and belts of any material coated with an insoluble polymeric material, mylar or release paper, and others. In one embodiment the film product is cut into strip of about 1×1.25 inches (typically 50 mg). Other dimensions may be preferred depending on the application.

Specific examples of flavored vitamin film products (multivitamin oral dosage forms containing water soluble and fat soluble vitamins) include:
1. Lemon
2. Watermelon
3. Bubble Gum
4. Strawberry
5. Orange
6. Blue raspberry

This list is obviously not limiting

Appendix A specification sheets—describe film properties and film ingredients for exemplary strawberry and watermelon flavored products.

Definitions:

In this specification the terms below should be construed to have the indicated meanings:
1. “Water soluble film”—a film comprising by water soluble materials, i.e. materials which dissolve in water
   a. Examples of film formers
      i. HPMC
      ii. CMC
      iii. Pectin
      iv. Alginates
      v. Others
      vi. Or any combination of above
2. “Base Solution”—a liquid mixture of ingredients that contain mostly water, water soluble or dispersible materials, and additives
3. “Active”—any material that has nutritional or medical value in the body after consumption
4. “Additives”
   a. materials that perform specific functions in finished film
   b. Example: sweeteners, preservatives, plasticizers, stabilizers, stiffening agents
5. “Stabilizing agents”—additives that protect emulsion by increasing the viscosity and minimizing oil from combining together and separating into two layers (oil and water layer)
   a. Examples of stabilizers
      i. Pectin
      ii. Carrageenan
      iii. Xanthan gum
      iv. Alginates
6. “Emulsifiers”—materials that stabilize emulsion by creating smaller oil droplet size during homogenization and keep oil in water
   a. Examples of emulsifiers
      i. Tween 80
      ii. Gum Arabic
      iii. HPMC
      iv. Carrageenans
      v. Xanthan gum
      vi. Gum Arabic
7. “Emulsify”—process of mixing at least two materials that do not mix well (e.g. oil and water) at high shear and breaking oil droplets into smaller droplets and dispersing them throughout the medium.
8. “Emulsion”—a mixture of at least two immiscible liquids, one material is contained within 2^{nd} material
   a. Example: oil (fats) and water

A skilled reader will readily appreciate that the invention should not be limited to specific apparatus configurations or method steps disclosed in this specific description of the preferred embodiment. Those skilled in the art will also recognize that the present invention has a broad range of applications, and the embodiments admit of a wide range of modifications, without departure from the inventive concepts.

While the foregoing has described what are considered to be the best mode and/or other preferred embodiments of the invention, it is understood that various modifications may be made therein and that the invention may be implemented in various forms and embodiments, and that it may be applied in numerous applications, only some of which have been described herein. It is intended by the following claims to claim any and all modifications and variations that fall within the true scope of the inventive concepts.

1. An oral dosage form comprising:
one or more vitamins in an amount greater than 15 dry weight percent; and
a polymer having film forming capabilities.

2. The oral dosage form according to claim 1 wherein the vitamins are present in an amount greater than 20 dry weight percent.

3. The oral dosage form according to claim 1 wherein the vitamins are present in an amount greater than 25 dry weight percent.

4. The oral dosage form according to claim 1 wherein the vitamins are present in an amount between 25 and 35 dry weight percent.

5. The oral dosage form according to claim 1 wherein the vitamins exceed 35 dry weight percent.

6. The oral dosage form of any of claims 1-5 wherein the oral dosage form further comprises at least one plasticizer.

7. The oral dosage form of any of claims 1-6 wherein the oral dosage form further comprises at least one stabilizer.

8. The oral dosage form of any of claims 1-7 wherein the oral dosage form further comprises at least one emulsifier.

9. The oral dosage form of any of claims 1-8 wherein the oral dosage form further comprises at least one surfactant.

10. The oral dosage form of any of claims 1-9 wherein the oral dosage form further comprises at least one flavoring.

11. An oral dosage form comprising:
one or more vitamins in an amount greater than 30 dry weight percent; and
a polymer having film forming capabilities.

12. An oral dosage form comprising:
at least one vitamin and/or mineral and flavorings sufficient to mask the taste of the vitamin and/or mineral; and
a polymer having film forming capabilities.

13. An oral dosage form comprising:
one or more vitamins in an amount greater than 20 dry weight percent;
a polymer in amount greater than 25 dry weight percent;
a flavoring sufficient to mask the taste of the vitamins; and
a polymer having film forming capabilities.

14. An oral dosage form comprising:
a polymer constituent in an amount between 25 and 35 dry weight percent;
at least one vitamin and/or mineral in an amount between 25 and 35 dry weight percent; and
at least one flavoring in an amount between 7 and 18 dry weight percent.

15. A method of forming a polymer film comprising vitamins and/or minerals, comprising:
preparing a vitamin emulsion;
preparing a polymer base;
adding the vitamin emulsion to the polymer base; and
casting to form the polymer film.

16. A method of forming a polymer film comprising vitamins and/or minerals, comprising:
preparing a flavor emulsion;
preparing a polymer base;
adding the flavor emulsion to the polymer base; and
casting to form the polymer film.

17. A method of forming a polymer film comprising vitamins and/or minerals, comprising:
preparing a vitamin emulsion;
preparing a flavor emulsion;
preparing a polymer base;
adding the vitamin and flavor emulsions to the polymer base; and
casting to form the polymer film.

18. A method of forming a polymer film comprising at least one vitamin and/or mineral, comprising:
preparing a vitamin-flavor emulsion;
preparing a polymer base; and
adding the vitamin-flavor emulsion to the polymer base.

19. A method of forming a polymer-based film product comprising multivitamins and/or minerals, comprising:
preparing a vitamin-flavor emulsion;
preparing a polymer base;
adding the vitamin-flavor emulsion to the polymer base.

20. A method of forming a polymer-based film product comprising at least one vitamin and/or mineral, comprising:
preparing a vitamin-flavor emulsion and preparing a polymer base;
adding the vitamin-flavor emulsion to the polymer base, wherein the vitamins are present in an amount between 20 and 40 dry weight percent and one or more flavorings are present in an amount between 5 and 20 dry weight percent.

21. The method according to claim 20 wherein the polymer constituent is present in an amount between 20 and 40 dry weight percent.

22. The method according to claim 20 or 21 wherein the vitamin amount to between 25 and 35 dry weight percent, the flavorings amount to between 7 and 15 dry weight percent, and the polymer base material amounts to between 25 and 35 dry weight percent.

23. The method according to claim 20 or 21 wherein the vitamin is present in an amount greater than 20 dry weight percent.

24. The method according to claim 20 or 21 wherein the vitamin is present in an amount greater than 25 dry weight percent.

25. The method according to claim 20 or 21 wherein the vitamin is present in an amount greater than 30 dry weight percent.

26. The method according to any of claims 20-25 wherein the film contains flavors in an amount greater than 7 dry weight percent.

27. The method according to any of claims 20-25 wherein the film contains flavors in an amount greater than 10 dry weight percent.

28. The method according to any of claims 20-25 wherein the film contains flavors in an amount greater than 13 dry weight percent.

29. The oral dosage form according to any of claims 1-14 wherein the oral dosage form is a breath freshening strip.

30. The method according to any of claims 15-28 wherein the method of forming a polymer film is a method for making a breath freshening strip.

31. The oral dosage form of any of claims 1-14 or 29 wherein the oral dosage form is dissolvable in the mouth or throat or buccal cavity.

32. An apparatus for forming a polymer film comprising vitamins and/or minerals, the apparatus comprising a first holder and mixer for holding and mixing a polymer base solution; and an emulsifier unit comprising a second holder and mixer for holding and mixing a vitamin-flavor emulsion, and an emulsifier in fluid communication with the second holder to emulsify the emulsion, the first holder in fluid communication with the emulsifying unit to mix together the polymer base solution with the emulsion to form a solution for coating onto a coating surface to form the polymer film.

33. The apparatus of claim 32 wherein the first and second mixers are high shear mixers.
